(19) United States
(12) Patent Application Publication
(10) Pub. No.: US 2008/0262085 A1
(43) Pub. Date: Oct. 23, 2008

(54) Method For Restructuring Keratin Fibers

(75) Inventors: Sabine Kainz, Moers (DE); Ursula Huchel, Koln (DE); Swenja Kalischke, Rheurdt (DE)

Correspondence Address: JEFFER, MANGELS, BUTLER & MARMARO, LLP, 1900 AVENUE OF THE STARS, 7TH FLOOR, LOS ANGELES, CA, 90067, US

(73) Assignee: HENKEL KOMMANDITGESELLSCHAFT AUF AKTIEN, Dusseldorf (DE)

(21) Appl. No.: 11/569,123

(22) PCT Filed: Apr. 8, 2005
(86) PCT No.: PCT/EP2005/003696

§ 371 Date: Nov. 14, 2006

(30) Foreign Application Priority Data
May 18, 2004 (DE) DE 102004024506.1

(51) Int. Cl. A61K 8/46

(52) U.S. Cl. 514/562

(57) ABSTRACT

The invention relates to a method for restructuring keratin fibers during which a keratin fiber is brought into contact with cystine and with at least one dicarboxylic acid having 2 to 10 carbon atoms. The invention also relates to preparations for use in this method.

The invention relates to a method for restructuring keratinic fibers, in which method a keratin fiber is brought into contact with cystine and with at least one dicarboxylic acid having 2 to 10 carbon atoms. The invention further relates to preparations for application in said methods.

Keratinic fibers, in particular hair, have great significance in everyday life as a permanent constituent of the human body and as an essential constituent of human clothing and domestic textiles. Treatment with washing, cleaning, styling, and dyeing products for cleaning and conformation purposes, and exposure to environmental influences such as ozone, salt water, chlorinated water, IR, UV, and thermal radiation (blow-drying), result over time in cumulative damage to the fibers and thus to a diminution in their quality. For example, both the cleaning of hair with shampoos and decorative conformation of a hairstyle by dyeing or permanent-waving are interventions that influence the natural structure and properties of hair. The wet and dry combability, stability, fullness, gloss, and tactility of hair, for example, may be unsatisfactory after such a treatment. In the case of dyed hair, in particular, the retention of the dye on the hair may furthermore be unsatisfactory in a context of frequent hair washing, thus resulting in a gradual bleeding of the color.

Not least, the large quantity of stress on the hair resulting, for example, from dyeing or permanent waves, hair washing with shampoos, and environmental stresses, increase the importance of conditioning products having effects of sufficient strength that last as long as possible. Such conditioning agents influence the natural structure and properties of the hair. Subsequent to such treatments, for example, the wet and dry combability of the hair and its stability and fullness can be optimized, or the hair can be protected from increasing splitting.

It has therefore been usual for some time to subject the hair to a specific post-treatment. In this, the hair is treated, usually in the form of a rinse, with specific active substances, for example quaternary ammonium salts or specific polymers. Depending on the formulation, this treatment improves the hair's combability, stability and fullness, and decreases splitting.

Conditioning additives and film-formers are often also added to permanent-wave agents, but without thereby greatly improving the hair structure. Used for this purpose, for example, are high-molecular-weight polymers that are absorbed onto the uppermost layer of the skin and hair and produce there an external, subjectively perceptible improvement in hair softness. The structural damage in the interior of the hair, however, which is caused in the case of permanent waves especially by the reduction process, cannot thereby be decreased, since because of their size the substances cannot penetrate into the hair. The duration of the effects of the structure-improved additives is moreover often unsatisfactory, since they merely adhere to the surface of the hair.

There have been attempts to remedy this problem by polymerizing monomeric compounds directly on the hair. According to the teaching of U.S. Pat. No. 5,362,486, for example, certain urethane oligomers having terminal bisulfite or acrylate groups are applied onto the hair and then polymerized, forming adherent polymers in situ on the hair. This method favorably influences the surface properties of hair, for example volume, gloss, stability, combability, and resistance to the absorption of moisture and air pollutants and to loss of hair color. In the method, a radical polymerization takes place on the hair, i.e. the hair must be treated with radical formers such as, for example, benzoyl peroxide.

EP-A 1174112 discloses hair treatment agents that, in addition to an organic acid, contain as further mandatory constituents an organic solvent, a cationic surfactant, and a higher alcohol, and serve to repair pores in hairs.

The object of the present invention was to make available a method for the restructuring of keratinic fibers, which method exhibits advantages over the existing art and enables a sufficient effectiveness and duration of effect. The method was intended not only to be implementable under fiber-protecting conditions, but also to be physiologically unobjectionable, i.e. in particular to dispense with the use of toxicologically objectionable substances such as radical formers.

The object of the present invention is achieved by a method in which keratinic fibers are brought into contact with cystine and with at least one dicarboxylic acid having 2 to 10 carbon atoms.

It has been found, surprisingly, that with the aid of a combination of cystine and at least one dicarboxylic acid having 2 to 10 carbon atoms, the tensile elongation properties of keratinic fibers can be improved and their strength can be increased, with no damage occurring to the fibers.

DE-A 100 51 774 describes the use of short-chain carboxylic acids having a molecular weight of less than 750 as an active substance for restructuring keratinic fibers in cosmetic agents. The simultaneous use of cystine is neither described nor suggested therein, and neither are any particular effects of such a combination.

A first subject of the present invention is therefore a method for restructuring keratinic fibers, in which method a keratin fiber is brought into contact with cystine and with at least one dicarboxylic acid having 2 to 10 carbon atoms.

“Keratinic fibers” are to be understood according to the present invention as furs, wool, feathers, silk, and hairs, but in particular human hair.

It has been found in the context of the present invention that with the use of the method according to the present invention, the internal and external structure of keratinic fibers can be modified, in other words that a restructuring of keratinic fibers is made possible. “Restructuring” is understood for purposes of the present invention as, in particular, a fiber reinforcement, an increase in breaking load, and/or a decrease in the damage to keratinic fibers resulting from a wide variety of influences. The restoration of natural strength, for example, plays an essential role here. Restructured fibers may be notable, for example, for elevated breaking load, elevated strength, elevated elasticity, and/or elevated volume, which can be manifested in a hairstyle, for example, as greater fullness. They may furthermore exhibit improved gloss, improved softness, and/or easier combability.

The method according to the present invention serves to strengthen, protect, and repair keratinic fibers, and is very particularly suitable for improving hair structure and/or for reinforcing human hair. In particular, fiber properties such as strength, elasticity, or volume are positively influenced, in the direction of an increase in those properties. The method is furthermore suitable for styling purposes, such as shaping and shape retention.

The method according to the present invention is further suitable for protecting fibers from the damaging influence of light.

The method according to the present invention requires no toxicologically objectionable substances such as, for example, radical formers or radicals that occur as intermediaries.

A “dicarboxylic acid” is to be understood, for purposes of the invention, as a compound having at least two carboxyl groups, i.e., for example, also including a compound having three or more carboxyl groups.

In a preferred embodiment of the invention, however, the dicarboxylic acid used in the method according to the present invention contains two and no more, i.e. exactly two, carboxyl groups.

In preferred embodiments of the invention, dicarboxylic acids are used that are selected from the group constituted by HOOC—(CH₂)ₙ—COOH dicarboxylic acids where n=0 to 8, maleic acid, fumaric acid, sorbic acid, and mixtures of these substances. The HOOC—(CH₂)ₙ—COOH dicarboxylic acids where n=1 to 8 include, for example, oxalic acid, malonic acid, succinic acid, glutaric acid, adipic acid, pimelic acid and azelaic acid.

In a particularly preferred embodiment of the invention, succinic acid is used as a dicarboxylic acid.

In the method according to the present invention, cystine and the at least one dicarboxylic acid are used at a weight ratio from 99 to 1 to 1 to 99, preferably 10 to 1 to 1 to 10, and in particular 2 to 1 to 1 to 2.

A further subject of the invention is the use of a combination of cystine and at least one dicarboxylic acid having 2 to 10 carbon atoms for the restructuring of keratinic fibers, in particular of hair, the restructuring encompassing in particular a fiber reinforcement.

The present invention furthermore relates to a fiber, in particular a keratinic fiber, that is obtainable by way of the method described above.

The temperature upon implementation of contact between the dicarboxylic acid and the fiber is preferably in a range from approximately 15 to approximately 40° C.

The implementation of contact between the fiber and cystine and the dicarboxylic acid can be accomplished in such a way that further substances are additionally added. These substances are preferably selected so that they form a carrier, suitable for treatment of the fiber, for the cystine and/or the dicarboxylic acid.

The implementation of contact can be accomplished in such a way that the fiber is brought into contact successively with at least two preparations, of which one contains cystine and a further contains at least one dicarboxylic acid.

The method according to the present invention is preferably carried out so that a fiber is brought into contact with a preparation that contains both cystine and at least one dicarboxylic acid.

In the case in which the fiber is a hair, the substances present in the preparation in addition to cystine and/or the dicarboxylic acid preferably form a composition of the kind commonly known to one skilled in the art of hair cosmetics as a “waving agent.”

The carriers for the preparations utilized in the method according to the present invention can be solid, liquid, gelled, or pasty. They are preferably selected from aqueous systems, natural or synthetic oils, water-in-oil or oil-in-water emulsions. Systems of this kind, and methods for their manufacture, are known in the existing art, to which reference is herewith made. The preparations can be formulated as a creme, gel, or liquid. It is furthermore possible to package the agents in the form of foam aerosols that are loaded, with a liquefied gas such as, for example, propane/butane mixtures, nitrogen, CO₂, air, NO₂, dimethyl ether, chlorofluorocarbon propellants, or mixtures thereof, into aerosol containers having a foam valve. The individual components of the method according to the present invention are preferably utilized as a creme, gel, or liquid. The preparations utilized according to the present invention can furthermore be present in two or more phases. Two-phase and multiple-phase systems are systems in which at least two separate, continuous phases are present. In such systems, for example, an aqueous phase and one or more, for example two, mutually immiscible nonaqueous phases can be present separately from one another. Also possible, for example, are a water-in-oil emulsion and an aqueous phase present separately therefrom, or a water-in-oil emulsion and an aqueous phase present separately therefrom.

The subject matter of the invention is furthermore a preparation for use in the method according to the present invention. The preparation is preferably aqueous and contains
(a) 0.01 to 20 wt % cystine and
(b) 0.01 to 20 wt % of at least one dicarboxylic acid having 2 to 10 carbon atoms, as defined in the preceding text,
   based in each case on the total weight of the preparation.

The active substances described below can advantageously be used as further constituents of the preparations utilized in the method according to the present invention.

Surfactants are suitable as active substances. The term “surfactants” is understood as surface-active substances that form adsorption layers at surface and interfaces, or can aggregate in volume phases to form micelle colloids or lyotropic mesophases. A distinction is made among anionic surfactants, made up of a hydrophobic radical and a negatively charged hydrophilic head group; amphoteric surfactants, that carry both a negative and a compensating positive charge; cationic surfactants, that comprise a positively charged hydrophilic group in addition to a hydrophobic radical; and nonionic surfactants, which comprise no charges but have strong dipole moments, and are highly hydrated in aqueous solution. More-detailed definitions and properties of surfactants may be found in H.-D. Dörfler, Grenzflächen- und Kolloidchemie [Interfacial and colloid chemistry], VCH Verlagsgesellschaft mbH, Weinheim, 1994. The definition of terms reproduced above is found on pp. 190 ff. of that document.

All anionic surface-active substances suitable for use on the human body are suitable in principle as anionic surfactants in preparations according to the present invention. These substances are characterized by a water-solubility-creating anionic group such as, for example, a carboxylate, sulfate, sulfonate, or phosphate group, and a lipophilic alkyl group having approximately 8 to 30 C atoms. Glycol or polyglycol ether groups, ester, ether, and amide groups, and hydroxyl groups can additionally be contained in the molecule. Examples of suitable anionic surfactants are, in each case in the form of the sodium, potassium, and ammonium as well as mono-, di-, and trialkanolammonium salts having 2 or 3 C atoms in the alkanol group:
linear and branched fatty acids having 8 to 30 C atoms (soaps);
ethercarboxylic acids of the formula R—O—(CH₂—CH₂O)ₓ—CH₂—COOH, in which R is a linear alkyl group having 8 to 30 C atoms and x=0 or is 1 to 16;
acylsarcosides having 8 to 24 C atoms in the acyl group;
acyltaurides having 8 to 24 C atoms in the acyl group;
acylisethionates having 8 to 24 C atoms in the acyl group;
sulfosuccinic acid mono- and -dialkyl esters having 8 to 24 C atoms in the alkyl group, and sulfosuccinic acid monoalkylpolyoxyethyl esters having 8 to 24 C atoms in the alkyl group and 1 to 6 oxyethyl groups;
linear alkanesulfonates having 8 to 24 C atoms;
linear alpha-olefinsulfonates having 8 to 24 C atoms;
alpha-sulfofatty acid methyl esters of fatty acids having 8 to 30 C atoms;
alkyl sulfates and alkylpolyglycol ether sulfates of the formula R—O(CH₂—CH₂O)ₓ—OSO₃H, in which R is a preferably linear alkyl group having 8 to 30 C atoms and x=0 or is 1 to 12;
mixtures of surface-active hydroxysulfonates according to DE A-37 25 030;
sulfated hydroxyalkylpolyethylene and/or hydroxyalkylenepropylene glycol ethers according to DE-A-37 23 354;
sulfonates of unsaturated fatty acids having 8 to 24 C atoms and 1 to 6 double bonds, according to DE-A-39 26 344;
esters of tartaric acid and citric acid with alcohols that represent addition products of approximately 2-15 molecules ethylene oxide and/or propylene oxide with fatty alcohols having 8 to 22 C atoms;
alkyl and/or alkenyl ether phosphates of formula (II)

R¹(OCH₂CH₂)ₙ—O—P(O)(OX)(OR²) (II)
in which R¹ preferably denotes an aliphatic hydrocarbon radical having 8 to 30 carbon atoms, R² denotes hydrogen, a (CH₂CH₂O)ₙR¹ radical, or X, n denotes numbers from 1 to 10, and X denotes hydrogen, and alkali or alkaline-earth metal, or NR³R⁴R⁵R⁶, where R³ to R⁶, independently of one another, denote hydrogen or a C1 to C4 hydrocarbon radical; sulfated fatty acid alkylene glycol esters of formula (III)

R⁷CO(AlkO)ₙSO₃M (III)
in which R⁷CO— denotes a linear or branched, aliphatic, saturated and/or unsaturated acyl radical having 6 to 22 C atoms, Alk denotes CH₂CH₂, CHCH₃CH₂, and/or CH₂CHCH₃, n denotes numbers from 0.5 to 5, and M denotes a cation, as described in DE-OS 197 36 906.5;
monoglyceride sulfates and monoglyceride ether sulfates of formula (IV)

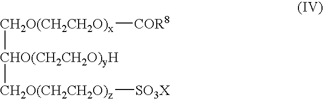

in which R⁸CO denotes a linear or branched acyl radical having 6 to 22 carbon atoms, x, y, and z in total denote 0 or numbers from 1 to 30, preferably 2 to 10, and X denotes an alkali or alkaline-earth metal. Typical examples of monoglyceride (ether) sulfates suitable for purposes of the invention are the reaction products of lauric acid monoglyceride, coconut fatty acid monoglyceride, palmitic acid monoglyceride, stearic acid monoglyceride, oleic acid monoglyceride, and tallow fatty acid monoglyceride, and their ethylene oxide adducts with sulfur trioxide or chlorosulfonic acid in the form of their sodium salts. Monoglyceride sulfates of formula (E1-III) are preferably used in which R⁸CO denotes a linear acyl radical having 8 to 18 carbon atoms, such as those that have been described, for example, in EP-B1 0 561 825, EP-B1 0 561 999, DE-A1 42 04 700, or by A. K. Biswas et al. in J. Am. Oil. Chem. Soc. 37, 171 (1960) and F. U. Ahmed in J. Am. Oil. Chem. Soc. 67, 8 (1990);
amide ether carboxylic acids such as those described in EP 0 690 044;
condensation products of C₈-C₃₀ fatty alcohols with protein hydrolysates and/or amino acids and their derivatives, known to one skilled in the art as protein fatty acid condensates, such as, for example, Lamepon® grades, Gluadin® grades, Hostapon® KCG, or the Amisoft® grades.

Preferred anionic surfactants are alkyl sulfates, alkylpolyglycol ether sulfates, and ethercarboxylic acids having 10 to 18 C atoms in the alkyl group and up to 12 glycol ether groups in the molecule, succinic acid mono- and dialkyl esters having 8 to 18 C atoms in the alkyl group, and sulfosuccinic acid monoalkylpolyoxyethyl esters having 8 to 18 C atoms in the alkyl group and 1 to 6 oxyethyl groups, monoglyceride sulfates, alkyl and alkenyl ether phosphates, and protein fatty acid condensates.

“Zwitterionic surfactants” refers to those surface-active compounds that contain in the molecule at least one quaternary ammonium group and at least one —COO⁽⁻⁾ or —SO₃⁽⁻⁾ group. Particularly suitable zwitterionic surfactants are the so-called betaines, such as the N-alkyl-N,N-dimethylammonium glycinates, for example cocalkyldimethylammonium glycinate, N-acylaminopropyl-N,N-dimethylammonium glycinates, for example cocacylaminopropyldimethylammonium glycinate, and 2-alkyl-3-carboxymethyl-3-hydroxyethylimidazolines having in each case 8 to 18 C-atoms in the alkyl or acyl group, as well as cocacylaminoethylhydroxyethylcarboxymethyl glycinate. A preferred zwitterionic surfactant is the fatty acid amide derivative known by the INCI name Cocamidopropyl Betaine.

“Ampholytic surfactants” are understood to be those surface-active compounds that contain in the molecule, in addition to a C₈-C₂₄ alkyl or acyl group, at least one free amino group and at least one —COOH or —SO₃H group, and are capable of forming internal salts. Examples of suitable ampholytic surfactants are N-alkylglycines, N-alkylpropionic acids, N-alkyl-aminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids, having in each case 8 to 24 C atoms in the alkyl group. Particularly preferred ampholytic surfactants are N-cocalkylaminopropionate, cocacylaminoethylaminopropionate, and C₁₂₋₁₈ acylsarcosine.

Nonionic surfactants contain as a hydrophilic group, for example, a polyol group, a polyalkylene glycol ether group, or a combination of a polyol and polyglycol ether group. Such compounds are, for example:
addition products of 2 to 50 mol ethylene oxide and/or 0 to 5 mol propylene oxide with linear fatty alcohols having 8 to 30 C atoms, with fatty acids having 8 to 30 C atoms, and with alkylphenols having 8 to 15 C atoms in the alkyl group;
addition products, end-capped with a methyl or C₂-C₆ alkyl radical, of 2 to 50 mol ethylene oxide and/or 0 to 5 mol propylene oxide with linear and branched fatty alcohols having 8 to 30 C atoms, with fatty acids having 8 to 30 C atoms, and with alkylphenols having 8 to 15 C atoms in the alkyl group, such as, for example, the grades obtainable under the commercial designations Dehydrol® LS, Dehydrol® LT (Cognis);
C₁₂-C₃₀ fatty acid mono- and diesters of addition products of 1 to 30 mol ethylene oxide with glycerol;
addition products of 5 to 60 mol ethylene oxide with castor oil and hardened castor oil;
polyol fatty acid esters such as, for example, the commercial product Hydagen® HSP (Cognis), or Sovermol grades (Cognis);
alkoxylated triglycerides;
alkoxylated fatty acid alkyl esters of formula (V)

R¹CO—(OCH₂CHR²)_{w}OR³ (V)
in which R¹CO denotes a linear or branched, saturated and/or unsaturated acyl radical having 6 to 22 carbon atoms, R² denotes hydrogen or methyl, R³ denotes linear or branched alkyl radicals having 1 to 4 carbon atoms, and w denotes numbers from 1 to 20;
amine oxides;
hydroxy mixed ethers, such as those described e.g. in DE-OS 197 38 866;
sorbitan fatty acid esters and addition products of ethylene oxide with sorbitan fatty acid esters, such as, for example, the polysorbates;
sugar fatty acid esters and addition products of ethylene oxide with sugar fatty acid esters;
addition products of ethylene oxide with fatty acid alkanolamides and fatty amines;
sugar surfactants of the alkyl and alkenyl oligoglycoside types, according to formula (VI)

R⁴O-[G]ₚ (VI)
in which R⁴ denotes an alkyl or alkenyl radical having 4 to 22 carbon atoms, G denotes a sugar radical having 5 or 6 carbon atoms, and p denotes numbers from 1 to 10. They can be obtained in accordance with the relevant methods of preparative organic chemistry. As a representative of the extensive literature, reference may be made here to the review articles of Biermann et al. in Starch/Stärke 45, 281 (1993), B. Salka in Cosm. Toil. 108, 89 (1993) and J. Kahre et al. in SÖFW-Journal Vol. 8, 598 (1995).
The alkyl and alkenyl oligoglycosides can be derived from aldoses or ketoses having 5 or 6 carbon atoms, preferably from glucose. The preferred alkyl or alkenyl oligoglycosides are thus alkyl and/or alkenyl oligoglucosides. The index number p in the general formula (E4-II) indicates the degree of oligomerization (DP), i.e. the distribution of mono- and oligoglycosides, and denotes a number between 1 and 10. Whereas p in the individual molecule must always be integral, and here can principally assume the values p=1 to 6, the value p for a specific alkyl oligoglycoside is an analytically ascertained calculated value, which usually represents a fractional number. Alkyl and/or alkenyl oligoglycosides having an average degree of oligomerization p of 1.1 to 3.0 are preferably used. In terms of applications engineering, those alkyl and/or alkenyl oligoglycosides whose degree of oligomerization is less than 1.7, and in particular between 1.2 and 1.4, are preferred. The alkyl or alkenyl radical R⁴ can be obtained from primary alcohols having 4 to 11, preferably 8 to 10 carbon atoms. Typical examples are butanol, hexanol, octanol, decanol, and undecyl alcohol as well as industrial mixtures thereof, such as those obtained, for example, upon hydrogenation of industrial fatty acid methyl esters or in the course of the hydrogenation of aldehydes from Roelen oxosynthesis. Preferred are alkyl oligoglucosides of chain length C₈-C₁₀ (DP=1 to 3), which occur as the first runnings upon distillational separation of industrial C₈-C₁₈ coconut oil alcohol and can be contaminated with a proportion of less than 6 wt % C₁₂ alcohol, and alkyl oligoglucosides based on industrial C_{9/11} oxoalcohols (DP=1 to 3). The alkyl or alkenyl radical R¹⁵ can furthermore be derived from primary alcohols having 12 to 22, preferably 12 to 14 carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and industrial mixtures thereof, which can be obtained as described above. Alkyl oligoglucosides based on hardened C_{12/14} cocalcohol having a DP of 1 to 3 are preferred.
sugar surfactants of the type of the fatty acid N-alkylpolyhydroxyalkylamides, a nonionic surfactant of formula (VII)

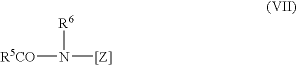

in which R⁵CO denotes an aliphatic acyl radical having 6 to 22 carbon atoms, R⁶ denotes hydrogen, an alkyl or hydroxyalkyl radical having 1 to 4 carbon atoms, and [Z] denotes a linear or branched polyhydroxyalkyl radical having 3 to 12 carbon atoms and 3 to 10 hydroxyl groups. The fatty acid N-alkylpolyhydroxyalkylamides are known substances that can usually be obtained by reductive amination of a reducing sugar with ammonia, an alkylamine, or an alkanolamine, and subsequent acylation with a fatty acid, a fatty acid alkyl ester, or a fatty acid chloride. With regard to the method for their manufacture, reference may be made to US patents U.S. Pat. No. 1,985,424, U.S. Pat. No. 2,016,962 and U.S. Pat. No. 2,703,798, and to International Patent Application WO 92/06984. A review of this topic by H. Kelkenberg is provided in Tens. Surf. Det. 25, 8 (1988). The fatty acid N-alkylpolyhydroxyalkylamides are preferably derived from reducing sugars having 5 or 6 carbon atoms, in particular from glucose. The preferred fatty acid N-alkylpolyhydroxyalkylamides therefore represent fatty acid N-alkylglucamides such as those reproduced by formula (VII):

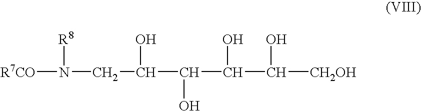

It is preferable to use as fatty acid N-alkylpolyhydroxyalkylamides glucamides of formula (E4-IV) in which R⁸ denotes hydrogen or an alkyl group, and R⁷CO denotes the alkyl radical of hexanoic acid, octanoic acid, decanoic acid, lauric acid, myristic acid, palmitic acid, palmoleic acid, stearic acid, isostearic acid, oleic acid, elaidic acid, petroselinic acid, linoleic acid, linolenic acid, arachidic acid, gadoleic acid, behenic acid, erucic acid, or industrial mixtures thereof. Particularly preferred are fatty acid N-alkylglucamides of formula (E4-IV) that are obtained by the reductive amination of glucose with methylamine and subsequent acylation with lauric acid or C_{12/14} coconut fatty acid, or a corresponding derivative. The polyhydroxyalkylamides can furthermore also derive from maltose and palatinose.

The alkylene oxide addition products with saturated linear fatty alcohols and fatty acids, having respectively 2 to 30 mol ethylene oxide per mol fatty alcohol or fatty acid, have proven to be preferred further nonionic surfactants. Preparations having outstanding properties are likewise obtained if they contain fatty acid esters of ethoxylated glycerol as nonionic surfactants.

These compounds are characterized by the following parameters: The alkyl radical R contains 6 to 22 carbon atoms and can be both linear and branched. Primary linear aliphatic radicals, and those methyl-branched in the 2-position, are preferred. Such alkyl radicals are, for example, 1-octyl, 1-decyl, 1-lauryl, 1-myristyl, 1-cetyl, and 1-stearyl. 1-octyl, 1-decyl, 1-lauryl, and 1-myristyl are particularly preferred. When so-called “oxo alcohols” are used as the initial materials, compounds having an odd number of carbon atoms in the alkyl chain predominate.

The sugar surfactants are also very particularly preferred nonionic surfactants. These can be contained in the preparations utilized according to the present invention preferably in quantities from 0.1-20 wt %, based on the entire preparation. Quantities from 0.5-15 wt % are preferred, and quantities from 0.5-7.5 wt % are very particularly preferred.

The compounds having alkyl groups used as surfactants can in each case be uniform substances. It is generally preferred, however, to begin with natural vegetable or animal raw materials when producing these substances, so that substance mixtures having different alkyl chain lengths, dependent on the particular material, are obtained.

In the case of the surfactants that represent addition products of ethylene oxide and/or propylene oxide with fatty alcohols, or derivatives of these addition products, both products having a “normal” homolog distribution and those having a restricted homolog distribution can be used. A “normal” homolog distribution is understood as mixtures of homologs that are obtained upon the reaction of fatty alcohol and alkylene oxide using alkali metals, alkali-metal hydroxides, or alkali-metal alcoholates as catalysts. Restricted homolog distributions, on the other hand, are obtained when, for example, hydrotalcites, alkaline-earth metal salts of ethercarboxylic acids, or alkaline-earth metal oxides, hydroxides, or alcoholates are used as catalysts. The use of products having a restricted homolog distribution can be preferred.

In a further embodiment of the invention, the preparation can contain a complex-forming agent, for example EDTA, NTA, β-alaninediacetic acid, a phosphonic acid, or mixtures of these substances.

Suitable as further active substances are polyols such as, for example, glycerol and partial glycerol ether, 2-ethyl-1,3-hexanediol, 1,3-butanediol, 1,4-butanediol, 1,2-propanediol, 1,3-propanediol, pentanedioles, for example 1,2-pentanediol, hexanedioles, for example 1,2-hexanediol or 1,6-hexanediol, dodecanediol, in particular 1,2-dodecanediol, neopentyl glycol, and ethylene glycol. In particular, 2-ethyl-1,3-hexanediol, 1,2-propanediol, 1,3-propanediol, and 1,3-butanediol have proven to be particularly well suited.

These polyols are contained in the preparations utilized according to the present invention in quantities preferably from 1 to 10, in particular 2 to 10 wt %, based on the entire preparation.

According to the present invention it is of course also possible to use alcohols that exhibit only limited miscibility with water, especially when multiple-phase systems are to be obtained.

“Limited miscibility with water” is understood to mean alcohols that are soluble in water at 20° C. at a proportion of no more than 10 wt % based on the mass of water.

Fatty substances can be used as further active substances. “Fatty substances” are to be understood as fatty acids, fatty alcohols, natural and synthetic waxes that can be present both in solid form and in liquid form in aqueous dispersion, and natural and synthetic cosmetic oil components.

Fatty acids that can be used are linear and/or branched, saturated and/or unsaturated fatty acids having 6 to 30 carbon atoms, in quantities from 0.1 to 15 wt % based on the entire agent. Fatty alcohols that can be used are saturated, mono- or polyunsaturated, branched or unbranched fatty alcohols having C₆-C₃₀ carbon atoms, in quantities from 0.1 to 30 wt % based on the entire preparation.

Natural and synthetic cosmetic oily substances that can be utilized according to the present invention as an active ingredient are, in particular:
vegetable oils. Examples of such oils are sunflower oil, olive oil, soybean oil, rapeseed oil, almond oil, jojoba oil, orange oil, wheat germ oil, peach-kernel oil, and the liquid components of coconut oil. Also suitable, however, are other triglyceride oils such as the liquid components of beef tallow, as well as synthetic triglyceride oils.
liquid paraffin oils, isoparaffin oils, and synthetic hydrocarbons, as well as di-n-alkyl ethers having a total of between 12 and 36 C atoms, in particular 12 to 24 carbon atoms, such as, for example, di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether, and n-hexyl-n-undecyl ether, as well as ditert-butyl ether, diisopentyl ether, di-3-ethyldecyl ether, tert-butyl-n-octyl ether, isopentyl-n-octyl ether, and 2-methylpentyl-n-octyl ether. The compounds 1,3-di-(2-ethylhexyl)cyclohexane (Cetiol® S) and di-n-octyl ether (Cetiol® OE), available as commercial products, may be preferred.

The quantity of the natural and synthetic cosmetic oily substances used in the preparations utilized according to the present invention is usually 0.1-30 wt % based on the entire preparation, preferably 0.1-20 wt %, and in particular 0.1-15 wt %.

The total quantity of oil and fat components in the preparations according to the present invention is usually 0.1-75 wt % based on the entire preparation. Quantities from 0.1 to 35 wt % are preferred according to the present invention.

It has furthermore been found that polymers are used advantageously in the context of the method according to the present invention. In a preferred embodiment, the preparations utilized according to the present invention therefore have polymers added to them, both cationic, nonionic, amphoteric, and nonionic polymers having proven effective.

“Cationic polymers” are to be understood as polymers that comprise in the main chain and/or side chain a group that can be “temporarily” or “permanently” cationic. According to the present invention, those polymers that comprise a cationic group regardless of the pH of the preparation are referred to as “permanently cationic.” These are, as a rule, polymers that contain a quaternary nitrogen atom, for example in the form of an ammonium group. Preferred cationic groups are quaternary ammonium groups. In particular, those polymers in which the quaternary ammonium group is bound via a C₁₋₄ hydrocarbon group to a main polymer chain made up of acrylic acid, methacrylic acid, or their derivatives, have proven to be particularly suitable.

Homopolymers of the general formula (IX),

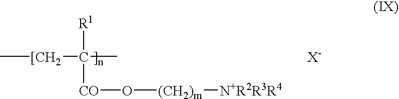

in which R¹=—H or —CH₃, R², R³ and R⁴ are selected, independently of one another, from C₁₋₄ alkyl, alkenyl, or hydroxyalkyl groups, m=1, 2, 3 or 4, n is a natural number, and X⁻ is a physiologically acceptable organic or inorganic ion, as well as copolymers made up substantially of the monomer units presented in formula (II) as well as nonionogenic monomer units, are particularly preferred cationic polymers. In the context of these polymers, those for which at least one of the following conditions apply are preferred according to the present invention:
R¹ denotes a methyl group
R², R³ and R⁴ denote methyl groups
m has a value of 2.

Possibilities as physiologically acceptable counterions X⁻ are, for example, halide ions, sulfate ions, phosphate ions, methosulfate ions, and organic ions such as lactate, citrate, tartrate, and acetate ions. Halide ions, in particular chloride, are preferred.

A particularly suitable homopolymer is the poly(methacryloyloxyethyltrimethylammonium chloride) (crosslinked, if desired) having the INCI name Polyquaternium-37. The crosslinking can be accomplished, if desired, with the aid of polyolefinically unsaturated compounds, for example divinylbenzene, tetraallyloxyethane, methylene-bisacrylamide, diallyl ether, polyallylpolyglyceryl ether, or allyl ethers of sugars or sugar derivatives such as erythritol, pentaerythritol, arabitol, mannitol, sorbitol, sucrose, or glucose. Methylene bisacrylamide is a preferred cross-linking agent.

The homopolymer is preferably used in the form of a nonaqueous polymer dispersion that should comprise a polymer proportion not less than 30 wt %. Such polymer dispersions are obtainable commercially under the designations Salcare® SC 95 (approx. 50% polymer proportion, further components: mineral oil (INCI name: Mineral Oil) and tridecylpolyoxypropylenepolyoxyethylene ether (INCI name: PPG-1-Trideceth-6)), and Salcare® SC 96 (approx. 50% polymer proportion, further components: mixture of diesters of propylene glycol with a mixture of caprylic and capric acid (INCI name: Propylene Glycol Dicaprylate/Dicaprate) and tridecylpolyoxypropylenepolyoxyethylene ether (INCI name: PPG-1-Trideceth-6)).

Copolymers having monomer units according to formula (II) preferably contain acrylamide, methacrylamide, acrylic acid C₁₋₄ alkyl esters, and methacrylic acid C₁₋₄ alkyl esters as nonionogenic monomer units. Of these nonionogenic monomers, acrylamide is particularly preferred. These copolymers as well, as in the case of the homopolymers described above, can be crosslinked. A copolymer preferred according to the present invention is the crosslinked copolymer of acrylamide and methacryloyloxyethyltrimethylammonium chloride. Such copolymers, in which the monomers are present at a weight ratio of approximately 20:80, are commercially obtainable as an approx. 50% nonaqueous polymer dispersion under the designation Salcare® SC 92.

Additional preferred cationic polymers are, for example:
quaternized cellulose derivatives such as those obtainable commercially under the designations Celquat® and Polymer JR®. The compounds Celquat® H 100, Celquat® L 200, and Polymer JR® 400 are preferred quaternized cellulose derivatives;
cationic alkylpolyglycosides according to DE Patent 44 13 686; cationized honey, for example the commercial product Honeyquat® 50;
cationic guar derivatives such as, in particular, the products marketed under the trade names Cosmedia® Guar and Jaguar®;
polysiloxanes having quaternary groups, such as, for example, the commercially obtainable products Q2-7224 (manufacturer: Dow Corning; a stabilized trimethylsilylamodimethicone), Dow Corning® 929 Emulsion (containing a hydroxylamino-modified silicone that is also referred to as amodimethicone), SM-2059 (manufacturer: General Electric), SLM-55067 (manufacturer: Wacker), and Abil®-Quat 3270 and 3272 (manufacturer: Th. Goldschmidt; diquaternary polydimethylsiloxanes, Quaternium-80);
polymeric dimethyldiallylammonium salts and their copolymers with esters and amides of acrylic acid and methacrylic acid. The products available commercially under the designations Merquat® 100 (poly(dimethyldiallylammonium chloride)) and Merquat® 550 (dimethyldiallylammonium chloride/acrylamide copolymer) are examples of such cationic polymers;
copolymers of vinylpyrrolidone with quaternized derivatives of dialkylaminoalkyl acrylate and methacrylate, such as, for example, vinylpyrrolidone/dimethylaminoethyl methacrylate copolymers quaternized with diethyl sulfate. Such compounds are obtainable commercially under the designations Gafquat® 734 and Gafquat® 755;
vinylpyrrolidone/vinylimidazolium methochloride copolymers, such as those offered under the designations Luviquat® FC 370, FC 550, FC 905, and HM 552;
quaternized poly(vinylalcohol); and
the polymers known under the designations Polyquaternium 2, Polyquaternium 17, Polyquaternium 18, and Polyquaternium 27, having quaternary nitrogen atoms in the main polymer chain.

The polymers known under the designations Polyquaternium-24 (commercial product, e.g. Quatrisoft® LM 200) can similarly be used as cationic polymers. Likewise usable according to the present invention are the copolymers of vinylpyrrolidone such as those available as the commercial products Copolymer 845 (manufacturer: ISP), Gaffix® VC 713 (manufacturer: ISP), Gafquat® ASCP 1011, Gafquat® HS 110, Luviquat® 8155, and Luviquat® MS 370.

Additional cationic polymers usable according to the present invention are the so-called “temporarily cationic” polymers. These polymers usually contain an amino group that is present at certain pH values as a quaternary ammonium group and therefore cationically. Chitosan and its derivatives, such as those readily available commercially, for example, under the commercial designations Hydagen® CMF, Hydagen® HCMF, Kytamer® PC, and Chitolam® NB/101, are, for example, preferred.

Cationic polymers that are preferred for use according to the present invention are cationic cellulose derivatives and chitosan and its derivatives, in particular the commercial products Polymer® JR 400, Hydagen® HCMF, and Kytamer® PC, cationic guar derivatives, cationic honey derivatives, in particular the commercial product Honeyquat® 50, cationic alkyl polyglycosides according to DE Patent 44 13 686, and polymers of the Polyquaternium-37 type.

The anionic polymers that can be utilized in the preparations of the method according to the present invention are anionic polymers that comprise carboxylate and/or sulfonate groups. Examples of anionic monomers of which such polymers are made up are acrylic acid, methacrylic acid, crotonic acid, maleic acid anhydride, and 2-acrylamido-2-methylpropanesulfonic acid. The acid groups can be present entirely or partially as a sodium, potassium, ammonium, mono- or triethanolammonium salt. Preferred monomers are 2-acrylamido-2-methylpropanesulfonic acid and acrylic acid.

Anionic polymers that contain 2-acrylamido-2-methylpropanesulfonic acid as a sole monomer or co-monomer have proven to be very particularly effective, in which context the sulfonic acid group can be present entirely or partially as a sodium, potassium, ammonium, mono- or triethanolammonium salt.

One such homopolymer of 2-acrylamido-2-methylpropanesulfonic acid is available commercially, for example, under the designation Rheothik® 11-80.

Within this embodiment, it may be preferred to use copolymers of at least one anionic monomer and at least one nonionogenic monomer. With regard to the anionic monomers, reference is made to the substances listed above. Preferred nonionogenic monomers are acrylamide, methacrylamide, acrylic acid ester, methacrylic acid ester, vinylpyrrolidone, vinyl ether, and vinyl ester.

Preferred anionic copolymers are acrylic acid/acrylamide copolymers and in particular polyacrylamide copolymers with sulfonic acid group-containing monomers. A particularly preferred anionic copolymer is made up of 70 to 55 mol % acrylamide and 30 to 45 mol % 2-acrylamido-2-methylpropanesulfonic acid, in which context the sulfonic acid group is present entirely or partially as a sodium, potassium, ammonium, mono-, or triethanolammonium salt. This copolymer can also be present in crosslinked form, polyolefinically unsaturated compounds such as tetraallyoxyethane, allylsucrose, allylpentaerythrite, and methylene bisacrylamide preferably being used as crosslinking agents.

Similarly preferred anionic homopolymers are uncrosslinked and crosslinked polyacrylic acids. Allyl ethers of pentaerythrite, of sucrose, and of propylene can be preferred crosslinking agents. Such compounds are obtainable commercially, for example, under the trademark Carbopol®.

Copolymers of maleic acid anhydride and methylvinyl ether, in particular those having crosslinks, are also well-suited polymers. A maleic acid/methylvinyl ether copolymer crosslinked with 1,9-decadiene is obtainable commercially under the designation Stabileze® QM.

Amphoteric polymers can furthermore be utilized as polymers in all aqueous preparations of the method according to the present invention. The term “amphoteric polymers” encompasses both those polymers that contain in the molecule both free amino groups and free —COOH or SO₃H groups and are capable of forming internal salts, and zwitterionic polymers that contain quaternary ammonium groups and —COO⁻ or —SO₃⁻ groups in the molecule, and those polymers that contain —COOH or SO₃H groups and quaternary ammonium groups.

One example of an amphopolymer usable according to the present invention is the acrylic resin obtainable under the name Amphomer®, which represents a copolymer of tert.-butylaminoethyl methacrylate, N-(1,1,3,3-tetramethylbutyl)acrylamide, and two or more monomers from the group of acrylic acid, methacrylic acid, and their simple esters.

Further amphoteric polymers usable according to the present invention are the compounds recited in GB Unexamined Application 2 104 091, EP Unexamined Application 47 714, EP Unexamined Application 217 274, EP Unexamined Application 283 817, and DE Unexamined Application 28 17 369.

Amphoteric polymers that are preferred for use are those polymerizates that are made up substantially of
a) monomers having quaternary ammonium groups of the general formula (X),

R¹—CH═CR²—CO-Z-(CₙH₂ₙ)—N⁽⁺⁾R³R⁴R⁵A⁽⁻⁾ (X)

in which R¹ and R², independently of one another, denote hydrogen or a methyl group and R³, R⁴, and R⁵, independently of one another, denote alkyl groups having 1 to 4 carbon atoms, Z denotes an NH group or an oxygen atom, n is a whole number from 2 to 5, and A⁽⁻⁾ is the anion of an organic or inorganic acid; and
b) monomeric carboxylic acids of the general formula (XI),

R⁶—CH═CR⁷—COOH (XI)

in which R⁶ and R⁷, independently of one another, are hydrogen or methyl groups.

These compounds can be used according to the present invention both directly and in a salt form obtained by neutralization of the polymerizate, for example using an alkali hydroxide. With regard to the details of the manufacture of these polymerizates, reference is made explicitly to the content of DE Unexamined Application 39 29 973. Those polymerizates in which monomers of type (a) are used in which R³, R⁴, and R⁵ are methyl groups, Z is an NH group, and A⁽⁻⁾ is a halide, methoxyfulfate, or ethoxysulfate ion, are very particularly preferred; acrylamidopropyltrimethylammonium chloride is a particularly preferred monomer (a). Acrylic acid is preferably utilized as monomer (b) for the aforesaid polymerizates.

Nonionogenic polymers can furthermore be contained in all aqueous preparations of the method according to the present invention. It can be preferred to utilize these in preparation (O).

Suitable nonionogenic polymers are, for example:
Vinylpyrrolidone/vinyl ester copolymers such as those marketed, for example, under the trademark Luviskol® (BASF). Luviskol® VA 64 and Luviskol® VA 73, which are each vinylpyrrolidone/vinyl acetate copolymers, are likewise preferred nonionic polymers.
Cellulose ethers, such as hydroxypropyl cellulose, hydroxyethyl cellulose, and methylhydroxypropyl cellulose, such as those marketed, for example, under the trademarks Culminal® and Benecel® (AQUALON).
Shellac;
Polyvinylpyrrolidones such as those marketed, for example, under the designation Luviskol® (BASF);
Siloxanes. These siloxanes can be both water-soluble and water-insoluble. Both volatile and nonvolatile siloxanes are suitable, “nonvolatile siloxanes” being understood as those compounds whose boiling point is above 200° C. at standard pressure. Preferred siloxanes are polydialkylsiloxanes such as, for example, polydimethylsiloxane, polyalklyarylsiloxanes such as, for example, polyphenylmethylsiloxane, ethoxylated polydialkylsiloxanes, and polydialkylsiloxanes that contain amine and/or hydroxy groups.
Glycosidically substituted silicones in accordance with EP 0612759 B1.

According to the present invention it is also possible for the preparations that are utilized to contain multiple, in particular two, different polymers of identical charge, and/or respectively one ionic and one amphoteric and/or nonionic polymer.

The polymers are contained in the preparations utilized according to the present invention preferably in quantities from 0.05 to 10 wt %, based on the entire preparation. Quantities from 0.1 to 5, in particular 0.1 to 3 wt %, are particularly preferred.

The preparations utilized according to the present invention can furthermore contain protein hydrolysates and/or, in addition to cystine, further amino acids and their derivatives. Protein hydrolysates are product mixtures obtained by the acid-, base-, or enzyme-catalyzed breakdown of proteins. The term “protein hydrolysates” is also understood according to the present invention to mean total hydrolysates as well as individual amino acids and their derivatives, as well as mixtures of different amino acids. Polymers constructed from amino acids and amino-acid derivatives are also to be understood under the term “protein hydrolysates” according to the present invention. Included among the latter are, for example, polyalanine, polyasparagine, polyserine, etc. Further examples of compounds usable according to the present invention are L-alanyl-L-proline, polyglycine, glycyl-L-glutamine, or D/L-methionine-S-methylsulfonium chloride. β-amino acids and their derivatives, such as β-alanine, anthranilic acid, or hippuric acid, can of course also be used according to the present invention. The molecular weight of the protein hydrolysates usable according to the present invention is between 75 (the molecular weight of glycine) and 200,000; preferably the molecular weight is 75 to 50,000 dalton, and very particularly preferably 75 to 20,000 dalton.

According to the present invention, protein hydrolysates of both plant and animal origin, or of marine or synthetic origin, can be used.

Animal protein hydrolysates are, for example, elastin, collagen, keratin, silk, and milk protein hydrolysates, which can also be present in the form of salts. Such products are marketed, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), Sericin (Pentapharm), and Kerasol® (Croda).

The use of protein hydrolysates of plant origin, e.g. soy-, almond-, bean-, potato-, and wheat-protein hydrolysates, is preferred according to the present invention. Such products are obtainable, for example, under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda), and Crotein® (Croda).

Although the use of protein hydrolysates as such is preferred, it is also optionally possible to use, instead of them, amino-acid mixtures obtained in different fashion. It is likewise possible to use derivatives of protein hydrolysates, for example in the form of their fatty acid condensation products. Such products are marketed, for example, under the designations Lamepon® (Cognis), Lexein® (Inolex), Crolastin® (Croda), Crosilk® (Croda), or Crotein® (Croda).

The protein hydrolysates or their derivatives are contained in the preparations utilized according to the present invention preferably in quantities from 0.1 to 10 wt %, based on the entire preparation. Quantities from 0.1 to 5 wt % are particularly preferred.

It is furthermore possible to utilize 2-pyrrolidinone-5-carboxylic acid and/or its derivatives in the preparations of the method according to the present invention. The sodium, potassium, calcium, magnesium or ammonium salts are preferred, in which context the ammonium ion carries, in addition to hydrogen, one to three C₁-C₄ alkyl groups. The sodium salt is very particularly preferred. The quantities used in the preparations according to the present invention are 0.05 to 10 wt % based on the entire preparation, particularly preferably 0.1 to 5 wt %, and in particular 0.1 to 3 wt %.

The use of vitamins, provitamins, and vitamin precursors, and their derivatives, has likewise proven advantageous.

Those vitamins, provitamins, and vitamin precursors that are usually allocated to the A, B, C, E, F, and H groups are preferred according to the present invention.

The group of substances referred to as vitamin A includes retinol (vitamin A₁) as well as 3,4-didehydroretinol (vitamin A₂). β-Carotene is the provitamin of retinol. Suitable vitamin A components according to the present invention are, for example, vitamin A acid and its esters, vitamin A aldehyde, and vitamin A alcohol, as well as its esters such as the palmitate and the acetate. The preparations utilized according to the present invention contain the vitamin A component preferably in quantities from 0.05 to 1 wt %, based on the entire preparation.

The vitamin B group or the vitamin B complex includes, among others:
Vitamin B₁ (thiamine)
Vitamin B₂ (riboflavin)
Vitamin B₃. The compounds nicotinic acid and nicotinic acid amide (niacinamide) are often listed under this designation. Nicotinic acid amide, which is contained in the preparations utilized according to the present invention preferably in quantities from 0.05 to 1 wt % based on the entire preparation, is preferred according to the present invention.
Vitamin B₅ (pantothenic acid, panthenol, and pantolactone). In the context of this group, panthenol and/or pantolactone are preferably used. Derivatives of panthenol that are usable according to the present invention are, in particular, the esters and ethers of panthenol as well as cationically derivatized panthenols. Individual representatives are, for example, panthenol triacetate, panthenol monoethyl ether and its monoacetate, and the cationic panthenol derivatives disclosed in WO 92/13829. The aforesaid compounds of the vitamin B₅ type are contained in the preparations utilized according to the present invention preferably in quantities from 0.05 to 10 wt % based on the entire preparation. Quantities from 0.1 to 5 wt % are particularly preferred.
Vitamin B₆ (pyridoxine, as well as pyridoxamine and pyridoxal).
Vitamin C (ascorbic acid). Vitamin C is used in the preparations utilized according to the present invention preferably in quantities from 0.1 to 3 wt % based on the entire preparation. Use in the form of the palmitic acid ester, the glucosides, or phosphates can be preferred. Use in combination with tocopherols can likewise be preferred.
Vitamin E (tocopherols, in particular α-tocopherol). Tocopherol and its derivatives, which include in particular the esters such as the acetate, nicotinate, phosphate, and succinate, are contained in the preparations utilized according to the present invention preferably in quantities from 0.05 to 1 wt % based on the entire preparation.
Vitamin F. The term “vitamin F” is usually understood to mean essential fatty acids, in particular linoleic acid, linolenic acid, and arachidonic acid.
Vitamin H. What is referred to as “vitamin H” is the compound (3aS,4S,6aR)-2-oxohexahydrothienol[3,4-d]-imidazol-4-valeric acid, for which the trivial name “biotin” has nevertheless now become established. Biotin is contained in the preparations utilized according to the present invention preferably in quantities from 0.0001 to 1.0 wt %, in particular in quantities from 0.001 to 0.01 wt %.

The preparations utilized according to the present invention preferably contain vitamins, provitamins, and vitamin precursors from groups A, B, E, and H.

Panthenol, pantolactone, pyridoxine and its derivatives, and nicotinic acid amide and biotin, are particularly preferred.

Lastly, plant extracts can be used in the preparations of the method according to the present invention.

These extracts are usually produced by extraction of the whole plants. In individual cases, however, it may also be preferred to produce the extracts exclusively from blossoms and/or leaves of the plants.

With regard to the plant extracts usable according to the present invention, reference is made in particular to the extracts listed in the table that begins on page 44 of the 3rd edition of the Leitfaden zur Inhaltsstoffdeklaration kosmetischer Mittel [Guidelines for declaring the ingredients of cosmetic agents], published by the Industrieverband Körperpflege- und Waschmittel e.V. [Federation of the Personal Hygiene and Washing Agents Industry] (IKW), Frankfurt.

Especially preferred according to the present invention are extracts from green tea, oak bark, nettle, hamamelis, hops, henna, chamomile, burdock root, horsetail, hawthorn, linden blossoms, almond, aloe vera, pine needles, horse chestnut, sandalwood, juniper, coconut, mango, apricot, lemon, wheat, kiwi fruit, melon, orange, grapefruit, salvia, rosemary, birch, mallow, lady's-smock, wild thyme, yarrow, thyme, lemon balm, restharrow, coltsfoot, hibiscus, meristem, ginseng, and ginger root.

The extracts from green tea, oak bark, nettle, hamamelis, hops, chamomile, burdock root, horsetail, linden blossoms, almond, aloe vera, coconut, mango, apricot, lemon, wheat, kiwi fruit, melon, orange, grapefruit, salvia, rosemary, birch, lady's-smock, wild thyme, yarrow, restharrow, meristem, ginseng, and ginger root are particularly preferred.

The extracts from green tea, almond, aloe vera, coconut, mango, apricot, lemon, wheat, kiwi fruit, and melon are very particularly suitable for the use according to the present invention.

Water, alcohol, and mixtures thereof can be utilized as extraction media for producing the aforesaid plant extracts. Among the preferred alcohols are the lower alcohols such as ethanol and isopropanol, but in particular polyvalent alcohols such as ethylene glycol and propylene glycol, both as the sole extraction medium and mixed with water. Plant extracts based on water/propylene glycol at a ratio from 1:10 to 10:1 have proven particularly suitable.

The plant extracts can be used according to the present invention both in pure and in diluted form. If they are used in diluted form, they usually contain approx. 2 to 80 wt % active substance and, as solvent, the extraction medium or extraction medium mixture used to obtain them.

It may furthermore be preferred to use, in the agents according to the present invention, mixtures of several, in particular two, different plant extracts.

It is furthermore preferred according to the present invention to use hydroxycarboxylic acids, and in this context in turn, in particular, the dihydroxy-, trihydroxy-, and polyhydroxycarboxylic acids, as well as the dihydroxy-, trihydroxy-, and polyhydroxydi-, tri-, and polycarboxylic acids. It has been shown in this context that in addition to the hydroxycarboxylic acids, the hydroxycarboxylic acid esters, as well as mixtures of hydroxycarboxylic acids and their esters and also polymeric hydroxycarboxylic acids and their esters, can be very particularly preferred. Preferred hydroxycarboxylic acid esters are, for example, full esters of glycolic acid, lactic acid, malic acid, tartaric acid, or citric acid. Further hydroxycarboxylic acid esters that are suitable in principle are esters of β-hydroxypropionic acid, tartronic acid, D-gluconic acid, saccharic acid, mucic acid, or glucuronic acid. Primary linear or branched aliphatic alcohols having 8 to 22 C atoms, i.e., for example, fatty alcohols or synthetic fatty alcohols, are suitable as alcohol components of these esters. The esters of C12-C15 fatty alcohols are particularly preferred in this context. Esters of this type are available commercially, e.g. under the trademark Cosmacol® of EniChem, Augusta Industriale. Particularly preferred polyhydroxypolycarboxylic acids are polylactic acid and polytartaric acid and their esters.

In a further preferred embodiment, emulsifiers are used in the preparations of the method according to the present invention. Emulsifiers cause the formation, at the phase interface, of water- or oil-stable adsorption layers that prevent the dispersed droplets from coalescing and therefore stabilize the emulsion. Emulsions are therefore, like surfactants, constructed from a hydrophobic and a hydrophilic molecule. Hydrophilic emulsifiers preferably form O/W emulsions, and hydrophobic emulsifiers preferably form W/O emulsions. An “emulsion” is to be understood as a droplet-like distribution (dispersion) of one liquid in another liquid, with the expenditure of energy to create stabilizing phase interfaces by means of surfactants. The selection of these emulsifying surfactants or emulsifiers is based on the substances to be dispersed and the respective external phase, and on the fineness of the emulsion particles. More-detailed definitions and properties of surfactants may be found in H.-D. Dörfler, Grenzflächen- und Kolloidchemie [Interfacial and colloid chemistry], VCH Verlagsgesellschaft mbH, Weinheim, 1994. Emulsifiers usable according to the present invention are, for example:
addition products of 4 to 30 mol ethylene oxide and/or 0 to 5 mol propylene oxide with linear fatty alcohols having 8 to 22 C atoms, with fatty acids having 12 to 22 C atoms, and with alkylphenols having 8 to 15 C atoms in the alkyl group;
C₁₂-C₂₂ fatty acid mono- and diesters of addition products of 1 to 30 mol ethylene oxide with polyols having 3 to 6 carbon atoms, in particular with glycerol;
ethylene oxide and polyglycerol addition products with methyl glucoside fatty acid esters, fatty acid alkanolamides, and fatty acid glucamides;
C₈-C₂₂ alkyl mono- and oligoglycosides and their ethyoxylated analogs, degrees of oligomerization from 1.1 to 5, in particular 1.2 to 2.0, and glucose as the sugar component, being preferred;
mixtures of alkyl (oligo)glucosides and fatty alcohols, for example the commercially available product Montanov® 68;
addition products of 5 to 60 mol ethylene oxide with castor oil and hardened castor oil;
partial esters of polyols having 3 to 6 carbon atoms with saturated fatty acids having 8 to 22 C atoms;
Sterols. “Sterols” are understood to mean a group of steroids that carry a hydroxyl group on the third C atom of the steroid structure and are isolated both from animal tissue (zoosterols) and from vegetable fats (phytosterols). Examples of zoosterols are cholesterol and lanosterol. Examples of suitable phytosterols are ergosterol, stigmasterol, and sitosterol. Sterols (the so-called mycosterols) are also isolated from fungi and yeasts. Phospholipids. These are understood to mean principally the glucose phospholipids, which are obtained e.g. as lecithins or phosphatidylcholines from for example, egg yolk or plant seeds (e.g. soybeans).
Fatty acid esters of sugars and sugar alcohols, such as sorbitol;
Polyglycerols and polyglycerol derivatives such as, for example, polyglycerol poly-12-hydroxystearate (commercial product Dehymuls® PGPH);
Linear and branched fatty acids having 8 to 30 C atoms, and their Na, K, ammonium, Ca, Mg, and Zn salts.

The preparations according to the present invention contain the emulsifiers preferably in quantities from 0.1 to 25 wt %, in particular 0.1 to 3 wt %, based on the entire preparation.

The preparations according to the present invention can preferably contain at least one nonionogenic emulsifier having an HLB value from 8 to 18, according to the definitions set forth in the Römpp-Lexikon Chemie [Römpp chemical dictionary] (J. Falbe, M. Regitz, eds.), 10th edition, Georg Thieme Verlag Stuttgart, New York (1997), page 1764. Nonionogenic emulsifiers having an HLB value from 10 to 15 may be particularly preferred according to the present invention.

Heterocyclic compounds such as, for example, derivatives of imidazole, pyrrolidine, piperidine, dioxolane, dioxane, morpholine, and piperazine can be used as further active substances. Also suitable are derivatives of these compounds such as, for example, the C₁₋₄ alkyl derivatives, C₁₋₄ hydroxyalkyl derivatives, and C₁₋₄ aminoalkyl derivatives. Preferred substituents, which can be positioned both on carbon atoms and on nitrogen atoms of the heterocyclic ring systems, are methyl, ethyl, β-hydroxyethyl, and β-aminoethyl groups. These derivatives preferably contain one or two of these substituents.

Derivatives of heterocyclic compounds that are preferred according to the present invention are, for example, 1-methylimidazole, 2-methylimidazole, 4(5)-methylimidazole, 1,2-dimethylimidazole, 2-ethylimidazole, 2-isopropylimidazole, n-methylpyrrolidone, 1-methylpiperidine, 4-methylpiperidine, 2-ethylpiperidine, 4-methylmorpholine, 4-(2-hydroxyethyl)morpholine, 1-ethylpiperazine, 1-(2-hydroxyethyl)piperazine, 1-(2-aminoethyl)piperazine. Further imidazole derivatives preferred according to the present invention are biotin, hydantoin, and benzimidazole.

Of these heterocyclic active substances, the mono- and dialkylimidazoles, biotin, and hydantoin are particularly preferred.

These heterocyclic compounds are contained in the preparations according to the present invention in quantities from 0.5 to 10 wt % based on the entire preparation. Quantities from 2 to 6 wt % have proven particularly suitable.

Further active substances that can be contained in all aqueous preparations utilized according to the present invention are, according to the present invention, amino acids and amino acid derivatives. From the group of the amino acids, arginine, citrulline, histidine, ornithine, and lysine have proven to be suitable according to the present invention. The amino acids can be used both as free amino acid and as salts, e.g. as hydrochlorides. In addition, oligopeptides made up of an average of 2-3 amino acids, and that have a high concentration (>50%, in particular >70%) of the aforesaid amino acids, have also proven to be usable according to the present invention.

Particularly preferred according to the present invention are arginine and its salts, and arginine-rich oligopeptides.

These amino acids or derivatives are contained in the preparations according to the present invention in quantities from 0.5 to 10 wt % based on the entire preparation. Quantities from 2 to 6 wt % have proven particularly suitable.

In addition, it may prove advantageous if penetration adjuvants and/or swelling agents are contained in the preparations according to the present invention. Included among these are, for example, urea and urea derivatives, guanidine and its derivatives, arginine and its derivatives, water glass, imidazole and its derivatives, histidine and its derivatives, benzyl alcohol, glycerol, glycol and glycol ethers, propylene glycol and propylene glycol ethers, for example propylene glycol monoethyl ether, carbonates, hydrogencarbonates, diols, and triols, and in particular 1,2-diols and 1,3-diols such as, for example, 1,2-propanediol, 1,2-pentanediol, 1,2-hexanediol, 1,2-dodecanediol, 1,3-propanediol, 1,6-hexanediol, 1,5-pentanediol, 1,4-butanediol. The penetration adjuvants and swelling agents are contained in the preparations utilized according to the present invention in quantities from 0.1 to 20 wt %, based on the entire preparation. Quantities from 01, to 10 wt % are preferred.

The preparations according to the present invention can also, especially in the case of waving lotions, contain waving-power-increasing components, in particular urea, imidazole, and the aforementioned diols. With regard to further information about such waving-power-increasing components, the reader is referred to the documents DE Unexamined Application 44 36 065 and EP-B1-363 057, to whose content reference is explicitly made.

The waving-power-increasing compounds can be contained in the preparations according to the present invention in quantities from 0.5 to 5 wt %, based on the entire preparation. Quantities from 1 to 4 wt % have proven to be sufficient, and these quantities are therefore particularly preferred.

In preparations according to the present invention that represent a fixing agent, in addition to at least one oxidizing agent such as, for example, hydrogen peroxide, a stabilizer usual for the stabilization of aqueous hydrogen peroxide preparations is preferably additionally used. The pH of such aqueous H₂O₂ preparations, which usually contain approximately 0.5 to 15 wt %, generally approximately 0.5-3 wt % in a ready-to-use state, H₂O₂, is preferably 2 to 6, in particular 2 to 4; it is adjusted by means of acids, preferably phosphoric acid, phosphonic acids, and/or dipicolinic acid. Bromate-based fixing agents contain the bromates usually in concentrations from 1 to 10 wt %, and the pH of the solutions is adjusted to 4 to 7. It can be particularly preferred according to the present invention to utilize fixing agent concentrates that are diluted with water before application.

It is additionally possible to carry out the oxidation with the aid of enzymes, the enzymes being used both to generate oxidizing per- compounds and to intensify the effect of a small quantity of an oxidizing agent that is present, or else enzymes are used that transfer electrons from suitable developer components (reducing agents) to atmospheric oxygen. Preferred in this context are oxidases such as tyrosinase, ascorbatoxidase, and laccase, but also glucoseoxidase, uricase, or pyruvatoxidase. Mention may also be made of the procedure of intensifying, by means of peroxidases, the effect of small quantities (e.g. 1% and less, based on the entire agent) of hydrogen peroxide.

The fixing agents according to the present invention can also be formulated as solids. They then contain the oxidizing agent in the form of a solid, e.g. potassium or sodium bromate. It is likewise possible, and preferred, to formulate the oxidizing agent as a two-component system. The two components, of which one is preferably a hydrogen peroxide solution or an aqueous solution of another oxidizing agent, and the other contains the remaining constituents, in particular care-providing substances and/or reducing agents, are once again not mixed until shortly before application.

In addition to the components already cited, the preparations utilized in step (b) of the method according to the present invention as surface-active compounds can contain cationic surfactants of the following types: quaternary ammonium compounds, esterquats, and amide amines. Preferred quaternary ammonium compounds are ammonium halides, in particular chlorides and bromides, such as alkyltrimethylammonium chlorides, dialkyldimethylammonium chlorides, and trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride, as well as the imidazolium compounds known under the INCI names Quaternium-27 and Quaternium-83. The long alkyl chains of the aforementioned surfactants preferably have 10 to 18 carbon atoms.

Esterquats are known substances that contain both at least one ester function and at least one quaternary ammonium group as structural elements. Preferred esterquats are quaternized ester salts of fatty acids with triethanolamine, quaternized ester salts of fatty acids with diethanolalkylamines, and quaternized ester salts of fatty acids with 1,2-dihydroxypropyldialkylamines. Such products are marketed, for example, under the trademarks Stepantex®, Dehyquart®, and Armocare®. The products Armocare® VGH-70, an N,N-bis(2-palmitoyloxyethyl)dimethylammonium chloride, as well as Dehyquart® F-75, Dehyquart® C-4046, Dehyquart® L80, and Dehyquart® AU-35, are examples of such esterquats.

The alkylamide amines are usually produced by amidation of natural or synthetic fatty acids and fatty acid cuts with dialkylaminoamines. A compound from this substance group that is particularly suitable according to the present invention is represented by the stearamidopropyldimethylamine available commercially under the designation Tegoamid® S 18.

The cationic surfactants are contained in the preparations utilized according to the present invention preferably in quantities from 0.05 to 10 wt % based on the entire agent. Quantities from 0.1 to 5 wt % are particularly preferred.

Additionally suitable as conditioning active substances are silicone oils and silicone gums, in particular dialkyl- and alkylarylsiloxanes, such as, for example, dimethylpolysiloxane and methylphenylpolysiloxane, as well as their alkoxylated and quaternized analogs. Examples of such silicones are the products marketed by Dow Corning under the designations DC 190, DC 200, and DC 1401, and the commercial product Fancorsil® LIM-1.

Particularly preferred according to the present invention as conditioning active substances are cationic silicone oils such as, for example, the commercially available products Q2-7224 (manufacturer: Dow Corning; a stabilized trimethylsilylamodimethicone), Dow Corning 929 Emulsion (containing a hydroxylamino-modified silicone that is also referred to as amodimethicone), SM-2059 (manufacturer: General Electric), SLM-55067 (manufacturer: Wacker) and Abil®-Quat 3270 and 3272 (manufacturer: Th. Goldschmidt; diquaternary polydimethylsiloxanes, Quaternium-80). A suitable anionic silicone oil is the product Dow Corning®1784.

Additional active substances, adjuvants, and additives are, for example:
thickening agents such as agar-agar, guar gum, alginates, xanthan gum, gum arabic, karaya gum, locust bean flour, linseed gums, dextrans, cellulose derivatives, e.g. methyl cellulose, hydroxyalkyl cellulose, and carboxymethyl cellulose, starch fractions and derivatives such as amylose, amylopectin, and dextrins, clays such as e.g. bentonite, or entirely synthetic hydrocolloids such as e.g. poly(vinyl alcohol),
hair-conditioning compounds such as phospholipids, for example soy lecithin, egg lecithin, and kephalins, as well as silicone oils,
perfume oils, dimethyl isosorbide, and cyclodextrins,
solvents and solubilizers such as ethanol, isopropanol, ethylene glycol, propylene glycol, glycerol, and diethylene glycol,
fiber-structure-improving active substances, in particular mono-, di-, and oligosaccharides such as, for example, glucose, galactose, fructose, fruit sugar, and lactose,
conditioning active substances such as paraffin oils, vegetable oils, e.g. sunflower oil, orange oil, almond oil, wheat germ oil, and peach-kernel oil, and
quaternized amines such as methyl-1-alkylamidoethyl-2-alkylimidazolinium methosulfate,
defoamers such as silicones,
dyes to color the agent,
anti-dandruff ingredients such as piroctone olamine, zinc omadine, and climbazol,
active substances such as allantoin bisabolol,
cholesterol,
consistency agents such as sugar esters, polyol esters, or polyol alkyl ethers,
fats and waxes such as spermaceti, beeswax, montan wax, and paraffins,
fatty acid alkanolamides,
swelling and penetrating substances such as primary, secondary, and tertiary phosphates,
opacifiers such as latex, styrene/PVP and styrene/acrylamide copolymers
luster agents such as ethylene glycol mono- and distearate, as well as PEG-3 distearate,
pigments,
propellants such as propane-butane mixtures, N₂O, dimethyl ether, CO₂, and air,
antioxidants.

With regard to further optional components and the quantities of said components that are used, the reader is referred expressly to the relevant manuals known to one skilled in the art, e.g. the aforementioned monograph of K. H. Schrader.

The preparations according to the present invention can be used in hair-care agents such as shampoos, conditioning agents, rinses, aerosols, and gels, and in hair dyeing agents, or also in agents for textile or fiber treatment, in the form of washing agents, conditioners, impregnations, and finishes.

The examples below are intended to explain the invention further.

### EXAMPLES

Unless otherwise indicated, all percentages indicated are to be understood as wt %, and all quantity indications as parts by weight.

To illustrate the effects according the present invention, a combination of succinic acid and cystine was incorporated during a cold-waving process (hair type: Natural dark brown, code no. 6634, of the Alkinco company). The contact times in the permanent wave process remained unchanged. The treatment temperature was 32° C.

1. Measurement Apparatus

To demonstrate the effects according to the present invention, the stress values, gradients, modulus of elasticity, elongation at fracture, and stress at fracture of the wet hairs were determined using a tensile elongation unit of the Dia-Stron company (MTT 670). The cross-sectional area of the individual wet hairs was determined by non-contact projection measurement using laser technology known in the existing art. A UMD5000A universal dimension instrument of the Zimmer company was utilized for this purpose.

2. Statistical Evaluation

The t-test, a statistical evaluation with which measurement series are compared in two-sided paired fashion, yields percentage probabilities that the measurement series are different (differences: 90-95%=measurement series are approaching difference; >95%=measurement series are significantly different; >99%=measurement series are very significantly different).

3. Restructuring Experiments

3.1 Hair Treatment

Forty individual hairs were divided into two parts. One part was damaged with two cold waves; the other part was treated with two cold waves in which a combination according to the present invention of succinic acid and cystine, and, for comparison, only one of those substances, had previously been added to the reducing agent. Before the fracture curves were ascertained, all 80 individual hairs were subjected, while wet, to a determination of the hair cross-sectional area.

3.2 Treatment Steps:

The following treatment steps were used in the experiments below:
a) Thirty-minute application of a cold wave (7% TGA=thioglycolic acid, 0.3% Turpinal SL=1-hydroxyethane-1,1-diphosphonic acid, 3.5% (NH₄)₂CO₃, pH 8.4). The hairs were then rinsed with water for 5 minutes.
b) Thirty-minute application of a cold wave (17% ammonium thioglycolate (71%), 0.3% Turpinal SL, 2.5% ammonia (25%), 5% ammonium hydrogencarbonate, 1% Cremophor RH 40 (hydrogenated castor oil+40-45 ethylene oxide; INCI name: PEG-40 Hydrogenated Castor Oil, BASF), 1% Lamepon S (protein/coconut fatty acid condensate, potassium salt; INCI name: Potassium Cocoyl Hydrolyzed Collagen, active substance content approx. 32%, COGNIS), 0.5% perfume, 0.1% Gluadin WQ (laurdimonium hydroxypropyl hydrolyzed wheat protein), 0.1% Merquat 100 (polydimethyldiallylammonium chloride), water to make 100%). The hairs were then rinsed out with water for 5 minutes.
c) Ten-minute application of fixing agent (2% H₂O₂, 1% Turpinal SL, pH 4.0). The hairs were then rinsed with water for 5 minutes.
d) Ten-minute application of fixing agent (5% H₂O₂, 0.2% NH₃, 1.7% Turpinal SL, 6% Texapon NSO UP (sodium lauryl ether sulfate), deionized water to make 100%). The hairs were then rinsed out with water for 5 minutes.
e) Leave hairs for at least 24 hours at room temperature (approx. 20° C.).
f) Measure cross sections of individual wet hairs.
g) Determine fracture values of individual wet hairs.

3.3 Results of Cold Wave with 1% Cystine

### Reference Example

Two permanent waves as described in 3.2: steps a), c), e); repeat steps a), c), e); then f) and g).

### Comparative Example

Similar treatment, 1% L-cystine having been added to the cold wave used in step a).

| | | | | |
|---|---|---|---|---|
| | | Stress | | |
| Elastic | Elastic | (plateau | Stress at 15% | Work at 15% |
| E-modulus | gradient | region) | elongation | elongation |
| [N/m²] | [N/mm] | [N/μm²] | [N/μm²] | [J] |

Reference example

| | | | | |
|---|---|---|---|---|
| 7.07E+08 | 1.08E−01 | 2.46E−05 | 2.33E−05 | 4.26E−04 |

Comparative example with 1% cystine

| | | | | |
|---|---|---|---|---|
| 7.81E+08 | 1.22E−01 | 2.62E−05 | 2.54E−05 | 4.80E−04 |

t-test, paired two-sided

| | | | | |
|---|---|---|---|---|
| significant | significant | significant | significant | significant |
| difference | difference | difference | difference | difference |

| | | |
|---|---|---|
| Stress at 25% | Work at 25% | Elongation at |
| elongation | elongation | fracture |
| [N/μm²] | [J] | [%] |

Reference example

| | | |
|---|---|---|
| 2.88E−05 | 8.08E−04 | 5.87E+01 |

Comparative example with 1% cystine

| | | |
|---|---|---|
| 3.15E−05 | 9.10E−04 | 5.45E+01 |

t-test, paired two-sided

| | | |
|---|---|---|
| significant | significant | significant |
| difference | difference | difference |

Result: the stress and work values for the plastic range, and the modulus of elasticity and gradient, are significantly different. A significant strengthening of the hair could be observed as a result of the permanent-wave treatment and the addition of 1% L-cystine; in other words, the addition of cystine to the waving agent allowed a reduction in the hair damage caused by the permanent wave.

3.4 Results of Cold Wave with 1% Cystine/Succinic Acid Mixture (1:1)

### Reference Example

Two permanent waves as described in 3.2: steps a), c), e); repeat steps a), c), e); then f) and g).

### Example According to the Present Invention

Similar treatment, 1% of a mixture of equal parts by weight L-cystine and succinic acid having been added to the cold wave used in step a).

| | | | | |
|---|---|---|---|---|
| | | Stress | Stress | Work |
| Elastic | Elastic | (plateau | at 15% | at 15% |
| E-modulus | gradient | region) | elongation | elongation |
| [N/m²] | [N/mm] | [N/μm²] | [N/μm²] | [J] |

Reference example

| | | | | |
|---|---|---|---|---|
| 8.57E+08 | 1.25E−01 | 3.04E−05 | 2.85E−05 | 4.85E−04 |

Example according to the present invention
with 1% succinic acid/L-cystine mixture

| | | | | |
|---|---|---|---|---|
| 9.30E+08 | 1.43E−01 | 3.19E−05 | 3.07E−05 | 5.52E−04 |

t-test, paired two-sided

| | | | | |
|---|---|---|---|---|
| significant | significant | significant | significant | significant |
| difference | difference | difference | difference | difference |

| | | |
|---|---|---|
| Stress | Work | |
| at 25% | at 25% | Elongation |
| elongation | elongation | at fracture |
| [N/μm²] | [J] | [%] |

Reference example

| | | |
|---|---|---|
| 3.52E−05 | 9.20E−04 | 5.98E+01 |

Example according to the present invention
with 1% succinic acid/L-cystine mixture

| | | |
|---|---|---|
| 3.74E−05 | 1.04E−03 | 5.59E+01 |

t-test, paired two-sided

| | | |
|---|---|---|
| significant | significant | significant |
| difference | difference | difference |

Result: The treatment with the permanent wave and the addition of a 1% succinic acid/cystine mixture produced significant hair strengthening. The modulus of elasticity and gradient, and the stress and work values for the plastic range, are significantly higher than for the reference permanent wave. Elongation at fraction is significantly less as compared with the reference, which likewise indicates restructuring.

This structural improvement is greater than that measured as a result of cystine alone (cf. experimental results in 3.3). This is indicated by a comparison of the experimental results in 3.3 with those in 3.4, with the aid of Scheffée covariance analysis based on multiple comparisons:

Statistical Evaluation:

To demonstrate the effect of the succinic acid/L-cystine mixture as compared with L-cystine, the two treatment groups were compared in terms of their initial values (cold-waved only) using Scheffée covariance analysis based on multiple comparisons.

The following significance levels were observed:

| | | Stress | Stress | Work |
|---|---|---|---|---|
| Elastic | Elastic | in plateau | at 15% | at 15% |
| E-modulus | gradient | region | elongation | elongation |
| approaching | approaching | significant | significant | approaching |
| difference | difference | difference | difference | difference |

| Stress | Work | |
|---|---|---|
| at 25% | at 25% | Elongation |
| elongation | elongation | at fraction |
| [N/μm²] | [J] | [%] |
| approaching | no difference | no difference |
| difference | | |

Overall result: The restructuring effect of L-cystine has already been observed. A significant restructuring can likewise be demonstrated when 1% succinic acid/L-cystine (1:1) is applied in the permanent wave. Covariance analysis demonstrated a significantly greater restructuring of the hairs resulting from the addition of a succinic acid/L-cystine mixture to the permanent wave, as compared with L-cystine, since the modulus of elasticity and gradient, and the stress and work values for the plastic range, are different. The combination of cystine and succinic acid may thus be considered to have a synergistic effect.

3.5. Results in a Commercially Available Cold Wave with 1% Succinic Acid:

### Reference Example

Two permanent waves with a commercial permanent wave as described in 3.2: steps b), d), e); repeat steps b), d), e); then f) and g).

### Comparative Example

Treatment similar to reference example, 1% succinic acid having been added to the cold wave used in step b).

| | | | | |
|---|---|---|---|---|
| | | Stress | Stress | Work |
| Elastic | Elastic | (plateau | at 15% | at 15% |
| E-modulus | gradient | region) | elongation | elongation |
| [N/m²] | [N/mm] | [N/μm²] | [N/μm²] | [J] |

Reference: damaged twice by cold wave

| | | | | |
|---|---|---|---|---|
| 8.09E+08 | 1.18E−01 | 2.83E−05 | 2.72E−05 | 4.65E−04 |

Comparative example given two cold
waves with 1% succinic acid added

| | | | | |
|---|---|---|---|---|
| 8.76E+08 | 1.26E−01 | 3.08E−05 | 2.91E−05 | 5.02E−04 |

t-test, two-sided paired

| | | | | |
|---|---|---|---|---|
| significant | significant | significant | significant | significant |
| difference | difference | difference | difference | difference |

| | | | |
|---|---|---|---|
| Stress | Work | | |
| at 25% | at 25% | Stress | |
| elongation | elongation | at fracture | Total work |
| [N/μm²] | [J] | [N/μm²] | [J] |

Reference: damaged twice by cold wave

| | | | |
|---|---|---|---|
| 3.49E−05 | 8.94E−04 | 1.22E−04 | 4.14E−03 |

Comparative example given two cold
waves with 1% succinic acid added

| | | | |
|---|---|---|---|
| 3.69E−05 | 9.58E−04 | 1.34E−04 | 4.60E−03 |

t-test, two-sided paired

| | | | |
|---|---|---|---|
| significant | significant | significant | significant |
| difference | difference | difference | difference |

Result: Succinic acid produces significant hair strengthening. When succinic acid is added to the permanent wave process, an increase in modulus of elasticity, gradient, stress values, and work is observed, from which an improvement in structure may be deduced.

3.6. Results in a Commercially Available Cold Wave Using a 1% Succinic Acid/Cystine Mixture

### Reference Example

Two permanent waves using a commercial permanent wave as described in 3.2: steps b), d), e); repeat steps b), d), e); then f) and g).

### Example According to the Present Invention

Treatment similar to reference example, a 1% succinic acid/L-cystine acid mixture 1:1 (equal parts by weight) having been added to the cold wave used in step b).

| | | | | |
|---|---|---|---|---|
| | | Stress | Stress | Work |
| Elastic | Elastic | (plateau | at 15% | at 15% |
| E-modulus | gradient | region) | elongation | elongation |
| [N/m²] | [N/mm] | [N/μm²] | [N/μm²] | [J] |

Reference: damaged twice by cold wave

| | | | | |
|---|---|---|---|---|
| 7.16E+08 | 1.02E−01 | 2.61E−05 | 2.46E−05 | 3.87E−04 |

Example according to the present invention
with 1% succinic acid/L-cystine mixture

| | | | | |
|---|---|---|---|---|
| 8.47E+08 | 1.18E−01 | 2.90E−05 | 2.75E−05 | 4.39E−04 |

t-test, two-sided paired

| | | | | |
|---|---|---|---|---|
| significant | significant | significant | significant | significant |
| difference | difference | difference | difference | difference |

| | |
|---|---|
| Stress | Work |
| at 25% | at 25% |
| elongation | elongation |
| [N/μm²] | [J] |

Reference: damaged twice by cold wave

| | |
|---|---|
| 3.11E−05 | 7.42E−04 |

Example according to the present invention
with 1% succinic acid/L-cystine mixture

| | |
|---|---|
| 3.39E−05 | 8.31E−04 |

t-test, two-sided paired

| | |
|---|---|
| significant | significant |
| difference | difference |

Result: Significant strengthening of the hair was observed as a result of the treatment according to the present invention. The stress and work values for the plastic range, and the modulus of elasticity and gradient, are significantly different. This hair strengthening is also significantly greater than that brought about by succinic acid alone (see example 3.5). A synergistic effect thus exists in the context of the mixture of succinic acid and cystine.

1. A method for restructuring keratinic fibers, in which method a keratin fiber is brought into contact with cystine and with at least one dicarboxylic acid having 2 to 10 carbon atoms.

2. The method according to claim 1, wherein the keratin fiber is a hair.

3. The method according to claim 1 or 2, wherein the restructuring encompasses a fiber reinforcement.

4. The method according to one of claims 1 to 3, wherein the dicarboxylic acid is selected from the group constituted by HOOC—(CH₂)ₙ—COOH dicarboxylic acids where n=0 to 8, maleic acid, fumaric acid, sorbic acid, and mixtures of these substances.

5. The method according to claim 4, wherein the dicarboxylic acid is succinic acid.

6. The method according to one of claims 1 to 5, wherein cystine and the at least one dicarboxylic acid are present at a weight ratio from 99 to 1 to 1 to 99,

7. The method according to claim 6, wherein cystine and the at least one dicarboxylic acid are present at a weight ratio from 10 to 1 to 1 to 10, but in particular from 2 to 1 to 1 to 2.

8. A preparation for use in a method according to one of claims 1 to 7, encompassing
(a) 0.01 to 20 wt % cystine
(b) 0.01 to 20 wt % of at least one dicarboxylic acid having 2 to 10 carbon atoms.

9. The preparation according to claim 8, wherein the dicarboxylic acid is selected from the group constituted by HOOC—(CH₂)ₙ—COOH dicarboxylic acids where n=0 to 8, maleic acid, fumaric acid, sorbic acid, and mixtures of these substances, and in particular denotes succinic acid.

10. A use of a combination of cystine and at least one dicarboxylic acid having 2 to 10 carbon atoms, for the restructuring of keratinic fibers, in particular of hair.

11. The use according to claim 10, wherein the restructuring encompasses a fiber reinforcement.
